(19) United States
(12) Patent Application Publication
(10) Pub. No.: US 2002/0073440 A1
(43) Pub. Date: Jun. 13, 2002

(54) METHOD FOR SCREENING AND TRANSGENIC MODEL

(76) Inventors: JAN TORNELL, VASTRA FROLUNDA (SE); JON KINDBLOM, GOTEBORG (SE); HAKAN WENNBO, GOTEBORG (SE); OLLE ISAKSSON, GOTEBORG (SE); GUNNAR NORSTEDT, BROMMA (SE)

Correspondence Address: BURNS DOANE SWECKER & MATHIS L L P, POST OFFICE BOX 1404, ALEXANDRIA, VA, 22313-1404, US

(21) Appl. No.: 09/367,272

(22) PCT Filed: Feb. 13, 1998
(86) PCT No.: PCT/SE98/00266

(30) Foreign Application Priority Data
Feb. 14, 1997 (SE) 9700527-6

(51) Int. Cl. A01K 67/027; A61K 38/24

(52) U.S. Cl. 800/13; 800/18; 514/12; 530/399; 536/23.4

(57) ABSTRACT

A method for the screening and identification of low molecular weight compounds which interact with lactogenic/somatogenic receptors is claimed. The method is characterized by the use of isolated tissues for in vitro cultivation as organ culture, primary cells, immortalized or transformed cells from transgenic animal that over-express hormones/receptors belonging to the somatic or lactogenic family. Also a transgenic non-human animal that over-express prolactin which cause phenotypic alterations notably in the prostate and/or mammary gland and use thereof is claimed.

### TECHNICAL FIELD OF THE INVENTION

The present invention relates to a method for the screening and identification of low molecular weight compounds which interact with lactogenic/somatogenic receptors and which is characterized by the use of isolated tissues for in vitro cultivation as organ culture, primary cells, immortalized or transformed cells from a transgenic non-human animal that over-express hormones/receptors belonging to the somatogenic or lactogenic family, e.g. prolactin (Pr1) and growth hormone (GH) receptors.

The invention also relates to a transgenic non-human animal that over-express prolactin which cause phenotypic alterations notably in the prostate and mammary gland and human e.g. growth hormone (GH) receptors and the use of the transgenic animal in the screening and identification method.

### BACKGROUND

Transgenic animals represent an important scientific tool to explore functions of specific genes in a physiological environment. Several examples exist where transgenic animals have been made that serve as useful models for human disease. The use of transgenic animals also include research in endocrinology and it has been well established that over-expression of growth hormone provides a model for acromegaly. In the creation of transgenic animals one can sometimes reveal more unexpected findings.

### DESCRIPTION OF THE INVENTION

The bases for the present invention is the unexpected finding that over-expression of prolactin (Pr1) cause a specific phenotype in prostatic and breast tissues. According to the present invention this finding can be used to establish a system where one can seek to find compounds that would circumvent the phenotype caused by the transgene. In the extension of this invention one can extrapolate into human systems and utilize the invention to find drugs that would function in humans.

As specified below, according to the present invention it has been found that a transgene that encode rat prolactin (rPr1) cause a specific phenotype to occur when expressed in transgenic mice. The phenotypic alterations include increased weight of thymus, spleen, kidney, testis, seminal vesicula and prostate without affecting body weight (see Abstract Vennbo et al; Growth Hormone Research Society, November 1996). The results are important for the understanding of the pathophysiology of Pr1. According to the present invention it is possible to use the animals as discussed above to screen for new drugs.

The present invention is based on the use of transgenic animals where the inventors of the present invention unexpectedly have found that the use of specific DNA constructs allows the generation of transgenic animals that exhibit a specific phenotype, these animals can be used as tools for screening and identification of low molecular weight compounds which interact with lactogenic/somatogenic receptors or in other ways inhibit or facilitate signals derived from lactogenic/somatogenic receptors and such use has not to our knowledge been reported earlier. According to the present invention it is also possible to grow cells derived from transgenic animals that are characterized by the expression of the transgene in question and that these cells subsequently can be used to screen compound libraries. The endpoint measurement may vary but need to be relevant for the effects of the transgene. Examples of cellular endpoint measurements per se is know in the art and include receptor binding/internalization assays, activation of intra cellular signals and proliferation assays.

The attached claims define the present invention. Examples, not intended to restrict the invention, are given below in order to illustrate the invention. The examples given are focused on the utility of rat Pr1 as a transgene of relevance but it is inferred that also human Pr1 and corresponding human Pr1 receptors may be used. The latter is relevant because of the pharmaceutical need for drugs that act on human receptors. In a similar type of experimental protocol the inventors also disclose the use of a human growth hormone receptor cDNA construction that is particularly suitable for the generation of an animal model that responds to human GH or analogues thereof.

### BRIEF DESCRIPTION OF THE DRAWINGS

In the examples below reference is made to the accompanying drawings on which:

FIG. 1 shows the Mt-rPRL-WBO2 plasmid.

FIG. 2 illustrates the correlation between the prostate wet weight and the serum testosterone levels in the PRL transgenic mice.

### EXAMPLES

### Example 1

Generation of an animal that over-express rat Pr1

An expression vector was constructed as outlined below. The key components in this vector is a metal-lothionein promoter followed by the rat Pr1 gene. This construct was injected into fertilized eggs using conventional techniques and offspring was analyzed using Southern blotting techniques. Positive off-spring were bred and later used to analyze phenotypic changes. It was shown that transgenic expression resulted in an increase in serum Pr1. It was also evident that certain tissues, notably prostate, increase in weight.

Materials and methods

Construction of the metallothionein promoter- rat prolactin plasmid

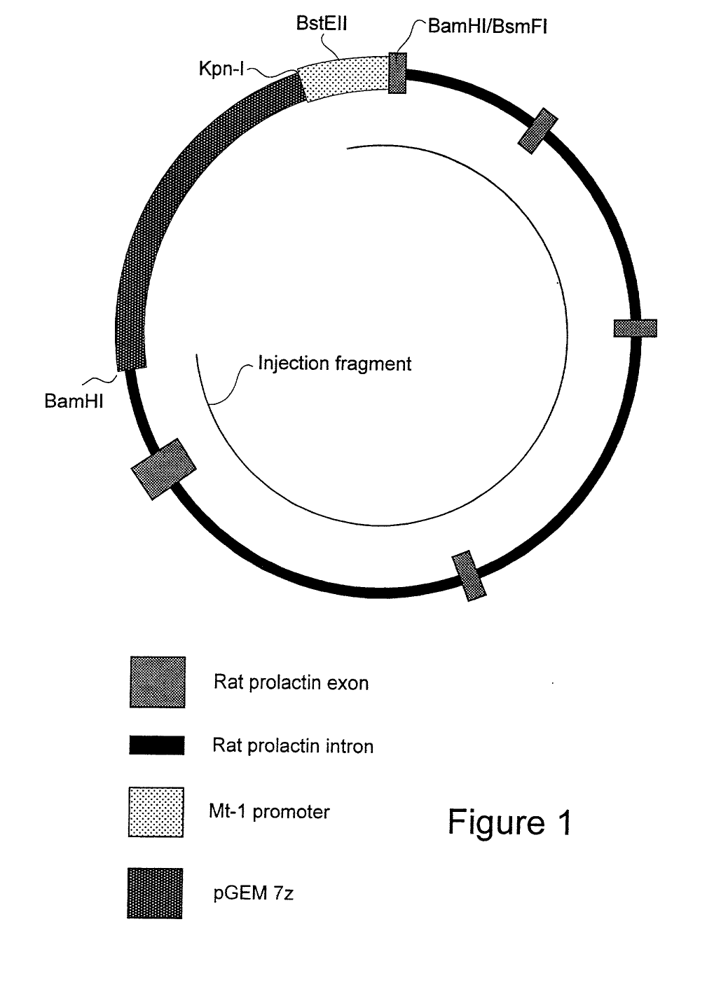

The rat PRL expression vector, Mt-rPRL-WBO2 was based on the pRPRL-HindIII A and B plasmids described earlier and the methallothionein-1 (Mt-1) promoter from MtbGH 2016 plasmid. The Mt-1 promoter was subcloned as a 650 bp fragment into a BsmF1 site 5′ of the start codon in the rat PRL gene inserted in a pGEM-7Z vector (Promega) resulting in the Mt-rPRL-WBO2 plasmid (FIG. 1). The metallothionein promoter, the rat prolactin gene and the injection fragment are indicated in FIG. 1. The Mt-1-rPRL fragment was excised by digestion with BstEII, located in the Mt-1 promoter, and BamHI located 3′ in the polylinker of pGEM-7Z. Transgenic mice were generated in C57BL/6JxCBA-f2 embryos by standard microinjection procedures. The DNA fragment to be injected was excised from the plasmid Mt-rPRL-WBO2 by restriction enzyme cleavage with BstEII and BamHI, separated by gel electrophoresis through a 0.7% agarose gel, cut out, isolated using isotachophoresis and precipitated with ethanol.

The bovine GH (bGH) DNA fragment was isolated from the plasmid MtbGH 2016 (generously provided by Dr. R. D. Palmiter) as a BstEII-EcoRI fragment, separated by gel electrophoresis through a 1% agarose gel, cut out, isolated using Genclean II kit (Bio 101). To identify transgenic animals DNA was extracted from 0.5 cm sections of tails from 3 weeks old mice by digestion with 400 mg of proteinase K in 0.6 ml of 1M urea, 100 mM NaCl, 50 mM Tris HCl (pH 8.0), 10 mM EDTA, 0.5% sodium dodecyl sulfate (SDS) at 55° C. for 16 h. The digested tails were freezed for 2 h in −70° C., then precipitated with isopropanol and washed with ethanol. The presence of the Mt-1 rPRL transgene was detected with PCR (94° C. for 5 min and 30 cycles of sequential incubations at 94° C. for 30 s, 54° C. for 30 s and 72° C. for 120 s) using one primer located in the Mt-promoter (5′-GCGAATGGGTTTACGGA-3′) and one in the rPRL gene (5′ CCATGAAGCTCCTGATGCT-3′). Mice that had integrated the bGH transgene were identified with PCR (the same incubation conditions as for rPRL) using the same Mt-promoter primer and one primer located in the bGH gene (5 CTCCAGGGACTGAGAACA-3). Tap water and pelleted food were freely available.

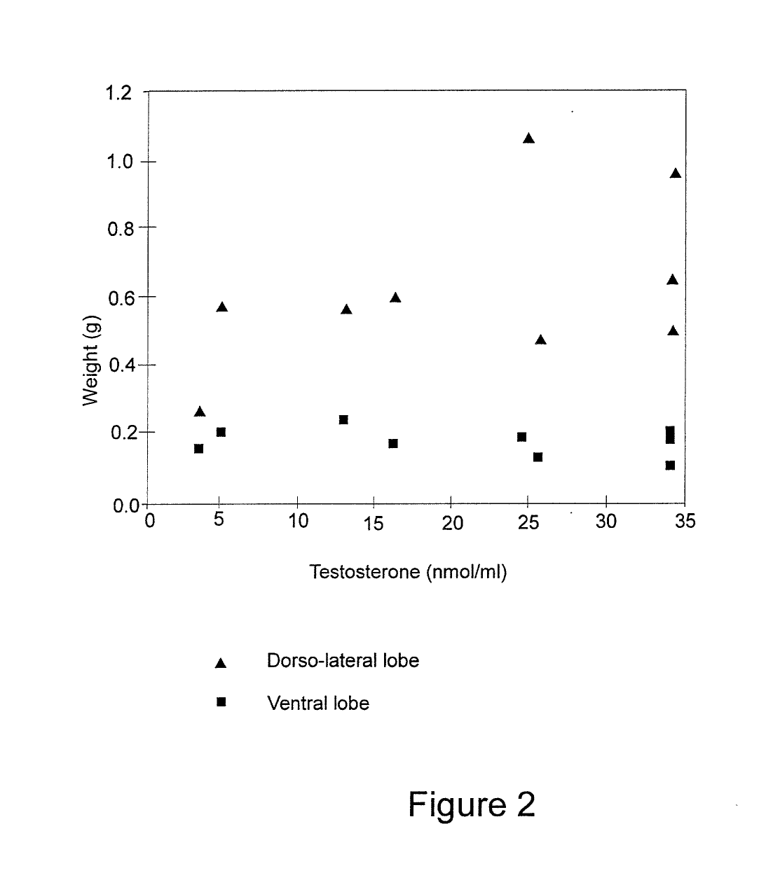

RNA analysis Total RNA was isolated from frozen tissues by acid guanidinium thiocyanate-phenol-chlorophorm extraction described by Chomczynski and Sacchi. Specific RNA was analyzed using a reverse transcriptase (RT)-PCR assay. The RT reaction was performed with 0.5 mg RNA as a template in the presence of 0.25 mg oligo-(dt) primer (Promega), 5 units AMV-RT (Promega), 20 units RNAsin (Promega) and dNTP (Pharmacia) at a final concentration of 1 mM per nucleotide. RT buffer (50 mM Tris-HCl; pH 8.3, 50 mM KCl, 10 mM MgCl₂, 0.5 mM spermidine and 10 mM DTT) was added to a total volume of 20 ml. After denaturation at 70° C. for 5 min and annealing in room temperature for 10 min the elongation was carried out for 60 min at 42° C. The RT reaction was terminated by heat inactivation (95° C. for 7.5 minutes). Rat PRL specific RNA was analyzed by amplifying an aliquot of cDNA by PCR (94° C. for 5 min and 30 cycles of sequential incubations at 94° C. for 30 s, 60° C. for 30 s and 72° C. for 120 s.) using a sense primer located in exon 4 (5-TCCATGAAGCTCCTGATG-T-3′) and an antisense primer located in exon 5 (5-GGATGGAAGTTGTGACCA-3′) specific for rat PRL (FIG. 2). The PCR products were analyzed by electrophoresis in 1% agarose gel. The size of the fragment amplified from spliced RNA should be 152 bp and that from unspliced RNA or contaminating DNA 1252 bp. The fragments were transferred to Hybond-N nylon membranes (Amersham) and the membranes were baked in 80° C. for 2 h and prehybridized in hybridization buffer (0.2 M NaH₂PO₄, pH 7.4, 8% SDS, 1 mM EDTA, 1% BSA fraction V) at 60° C. for 2 h. As probe a 823 bp PstI fragment containing the rat PRL cDNA was used, labeled with a random priming kit (Amersham) and P32dCTP.

The hybridization was carried out in the same buffer at 60° C. for 12-16 h and washed with 2×SSC, 0.5% SDS at 60° C. for 1-2 h and with 0.1×SSC, 0.1% SDS at 60° C. for 0.5-2 h. Mouse PRL specific RNA was analyzed by amplifying cDNA by PCR (94° C. for 5 min and 30 cycles of sequential incubations at 94° C. for 30 s, 56° C. for 30 s and 72° C. for 120 s.) using a sense primer located in exon 1 (5-GTCACCATGACCATGAAC-3) and an antisense primer located in exon 5 (5-GGATGGAAGTTGTG ACCA-3). The size of the fragment amplified from spliced RNA should be 558 bp. The transfer, hybridization, probe labeling and washing were carried out as above. The mouse PRL cDNA was amplified from mouse pituitary cDNA by the same protocol and primers used for detection of mouse PRL expression in prostate. The PCR fragment was subcloned into a pCRTMII vector (Invitrogen) and identified as mouse PRL by digestion with restriction enzymes. As probe the entire subcloned fragment from the vector was used. Specific RNA for the long form of the mouse PRL receptor was amplified by PCR (94° C. for 5 min and 30 cycles of sequential incubations at 94° C. for 30 s, 56° C. for 30 s and 72° C. for 120 s.) using a sense primer located in the extracellular part of the receptor (5 GACTCGCTGCAAGCCAGACC-3) and an antisense primer located in the intracellular part of the long form of the receptor (5-TGACCAGAGTCACTGTCAGG-3). The size of the fragment amplified from spliced RNA should be 440 bp. The transfer, hybridization, probe labeling and washing were carried out as above. An EcoRI-XhoI fragment of the plasmid 4A314 (R. Ball, Basel, Switzerland, unpublished data) containing the long form of the mouse PRL receptor cDNA was used as a probe. DNA content analysis. Total nucleic acids (TNA) were extracted by homogenization of frozen tissues in 1% sodiumdodesulphate (SDS), 20 mM Tris-HCl (pH 7.5) and 4 mM EDTA, followed by a 45 min digestion with proteinase-K in 45° C. and extraction with phenol-chloroform. The DNA content in the TNA preparations were measured with a fluorescence spectrophotometer (450 nm excitation and 555 nm emission) after addition of Hoecht s dye H 33258 (0.2 eeg/ml in 2 M NaCl, 1 mM EDTA and 10 mM Tris pH 7.4).

Measurement of rat prolactin: Serum levels of rat PRL were measured by rat prolactin RIA (Amersham U.K.) according to a protocol from the manufacturer. Mouse PRL does not cross react with the antibody raised against rat PRL according to the manufacturer. Serum was either collected from the mice tails in heparin coated glass capillaries or by heart puncture in heparin coated syringes when sacrificed. All samples were analyzed in duplicates.

Measurement of IGF-I: The IGF-I concentration in serum was determined by a radioimmunoassay after acid ethanol extraction according to the manufactures protocol (Nicols Institute Diagnostics, San Juan Capistrano Calif., USA) in a single assay.

Measurement of testosterone: Serum testosterone was measured by radioimmunoassay according to the manufactures protocol (ICN Biomedicals, Inc. Costa Mesa Calif., USA) Histology Tissue pieces were fixed in 4% paraformaldehyde in phosphate buffered saline (PBS; pH 7.4) overnight or longer, dehydrated and embedded in paraffin. Sections were stained in hematoxylin/eosin. Statistics Statistical differences were calculated using the Wilcoxon rank sum test. Significance levels less than 0.05 were considered significant.

Results

Generation of rat prolactin transgenic mice

The rat RPL gene has been described and contains 5 exons. The entire gene is approximately 11 kb and the cDNA 800 bp. In the Mt-rPRL-WBO2 plasmid used to generate rPRL transgenic mice the metallothionein-1 (Mt-1) promoter was inserted 33 basepairs upstream of the first exon into a BsmF1 site in the rPRL gene. The linerized fragment from the plasmid outlined in FIG. 1 was micro-injected into 250 mouse zygotes obtained after superovulation and implanted into 9 foster mothers resulting in 40 newborn mice. Three mice were identified as carrying the Mt-rPRL construct using PCR analysis. The founder animals, two females and one male were mated for establishing lines of transgenic mice. Transgenic lines were successfully established from the female founder animals mated to normal male mice. Male offspring from a bGH transgenic founder were used. Male mice from this line have serum levels of bGH higher than 5 times the normal peak values in mice.

The transgenic mice had elevated serum levels of rPRL The rPRL levels in the founder animals were evaluated by RIA analysis at 35 days of age. One female founder (L1) had very high levels of PRL (470 ng/ml) and the other female founder (L2) expressed the transgene at lower levels (11 ng/ml). The rPRL levels in the male founder (L3) was 32 ng/ml. Rat PRL levels were also measured in all the animals included in the study when sacrificed (Table 1). Expression levels of rPRL protein were stable over the life span of the animals. Offspring generated from transgenic line L1 showed consistently high serum rPRL-levels while offspring generated from L2-line expressed the transgene at lower levels (Table 1). Rat PRL transgenic mice developed marked enlargement of the prostate. The weight of prostate was examined when the animals were sacrificed at 10-15 months of age.

Histological examinations were carried out on all prostates. All prostates from PRL transgenic mice had a higher weight compared to age matched controls (Table 1). On average, the dorso-lateral lobes from the prostate were 20 times larger (wet weight) than the controls and the ventral parts were 9 times larger (wet weight) (Table 1). The bGH transgenic mice had 1.6-times larger dorso-lateral lobes of the prostate glands (Table 1) and also increased body weight (1.4 times larger than controls, data not shown). In contrast, the body weight of the prolactin transgenic mice were not increased (data not shown). The DNA content in the prostate glands were measured in 5 rPRL transgenic animals and 5 controls when sacrificed. The total DNA in the dorso-lateral lobe was increased 4.7 times (155 n 34 eeg DNA/prostate lobe vs. 33 n 5 eeg.DNA/prostate lobe in the controls) and in the ventral lobe 4.2 times (96 n 11 eeg DNA/prostate lobe vs. 23 n 5 eeg DNA/prostate lobe in the controls).

Histologically, all prostates from PRL transgenic mice showed hyperplasia and glands distended by secretion in contrast to non-transgenic animals. The proportion of stroma cells and connective tissue were increased in the rPRL transgenic animals compared to controls. Occasionally there were parts classified as adenomas and in some areas showing cellular atypia. Focal parts of chronic inflammation were also visible.

The rPRL transgene, the endogenous mPRL gene-and the PRLR were expressed in the prostate gland. Specific mRNA for the rPRL transgene was detected in both in the dorso-lateral part of the prostate and in the ventral lobe (Table 2)..Two of the lines (L1 and L2) expressed the transgene at higher levels compared to the third line when measured with the semi-quantitative method RT-PCR. In the normal animals, expression of the mouse PRL gene was detected in all parts of the prostate gland (Table 2). Also PRLR-specific mRNA was detected in the dorso-lateral lobe and the ventral lobe of the prostate (Table 2). Several different tissues were analyzed for the presence of mRNA corresponding to rPRL using a RT-PCR assay. The primers were selected in a way that the PCR reaction could not amplify cDNA corresponding to expression of the mouse PRL gene.

The transgene was expressed in the liver, kidney, pancreas, seminal vesicles, testis, thymus and the prostate gland. Expression of the PRL transgene in different organs were analyzed with RT-PCR as described in the material and methods. Tissue samples were collected from the line L1 at 14 months of age. An expected 152 bp fragment was detected in all organs analyzed. As a negative control the PCR reaction was run without any template added. Appropriate lanes from the same gel were selected.

Rat PRL transgenic mice had elevated levels of testosterone and of IGF-I. BGH transgenic mice had elevated levels of IGF-I. The serum levels of testosterone and IGF-I were measured when the animals were sacrificed. PRL transgenic-mice had higher levels of testosterone than controls (Table 1). The testosterone levels among the rPRL transgenic mice could not be correlated to the prostate weight in neither the dorso-lateral lobe or the ventral lobe as can be seen in FIG. 2. FIG. 2 shows the correlation between the prostate wet weight and the serum testosterone levels in the PRL transgenic mice. r=0.50 for serum testosterone correlated to the weight of the dorso-lateral prostate and r=0.20 for serum testosterone correlated to the weight of the ventral prostate. p>0.05

The IGF-I levels were elevated in the rPRL and the bGH transgenic animals compared to controls (Table 1).

### Conclusion

As exemplified above, prolactin transgenic animals may serve as a useful model for studying prostate hyperplasia. In addition to previously described transgenic models using expression of int-2 and large-T the present model represents a hormone dependent hyperplasia and might therefore be closer to the human patophysiology.

Other non-transgenic animal models for prostatic hyperplasia exist, e.g. testosterone induced BPH but our data indicates that prostatic growth in Pr1 transgenic mice could occur independently of at least serum concentrations of testosterone. In the human, the influence of PRL on prostate growth is not clear but the present study suggests that the question whether PRL is an important factor for development of prostate hyperplasia in man should be addressed. The use of transgenic animals may be instrumental in order to obtain and/or test e.g. inhibitors of Pr1 actions.

TABLE 1
Age when sacrificed, plasma levels of rat prolactin, testosterone and IGF-I and weight of
different lobes of the prostate gland in rat-prolactin (PRL) and bovine-growth hormone (bGH)
transgenic mice and normal control animals

| | | | | | | | | |
|---|---|---|---|---|---|---|---|---|
| | | | Dorso- | Weight | | Weight | | |
| | | Rat | lateral | compared to | Ventral | compared to | | |
| | Age | prolactin | prostate | normal | prostate | normal | Testosterone | IGF-I |
| Animal | (months) | (ng/ml) | (g) | (%) | (g) | (%) | (nmol/ml) | (ng/ml) |

Prolactin transgenic animals

| | | | | | | | | |
|---|---|---|---|---|---|---|---|---|
| 2 (L1) | 14 | 235 | 0.65 | 2000 | 0.19 | 943 | 34 | 569 |
| 14 (L1) | 10 | 260 | 0.97 | 2985 | 0.11 | 546 | 34 | 397 |
| 16 (L1) | 11 | 281 | 0.60 | 1846 | 0.17 | 844 | 16 | 513 |
| 1 (L2) | 14 | 17 | 0.56 | 1723 | 0.24 | 1190 | 13 | 238 |
| 3 (L2) | 14 | 16 | 1.07 | 3292 | 0.19 | 943 | 25 | 472 |
| 4 (L2) | 14 | 17 | 0.51 | 1569 | 0.21 | 1043 | 34 | 440 |
| 5 (L2) | 12 | 12 | 0.57 | 1754 | 0.20 | 993 | 5.0 | 358 |
| 7 (L2) | 14 | 15 | 0.48 | 1477 | 0.13 | 645 | 26 | 311 |
| 6 (L3) | 15 | 103 | 0.26 | 800 | 0.15 | 745 | 3.7 | 433 |
| Mean | | | 0.63**§§ | 1938 | 0.18**§§ | 877 | 21* | 415* |
| SEM | | | 0.08 | | 0.01 | | 4 | 34 |

Growth hormone transgenic animals

| | | | | | | | |
|---|---|---|---|---|---|---|---|
| 24 | 12 | 0.02 | 74 | 0.01 | 35 | 11 | 464 |
| 25 | 12 | 0.06 | 185 | 0.04 | 174 | 10 | 716 |
| 26 | 12 | 0.06 | 169 | 0.02 | 89 | 1 | 469 |
| 27 | 11 | 0.06 | 185 | 0.02 | 74 | 10 | 404 |
| 28 | 11 | 0.06 | 185 | 0.03 | 129 | 11 | 420 |
| Mean | | 0.05* | 159 | 0.02 | 100 | 8.3 | 495** |
| SEM | | 0.01 | | 0.005 | | 2.0 | 57 |

Normal animals

| | | | | | |
|---|---|---|---|---|---|
| 8 | 14 | 0.03 | 0.03 | 1.4 | 286 |
| 9 | 14 | 0.04 | 0.03 | 1.4 | 469 |
| 10 | 14 | 0.03 | 0.03 | 0.5 | 303 |
| 11 | 14 | 0.02 | 0.02 | 1.0 | 369 |
| 12 | 14 | 0.04 | 0.02 | 4.1 | 369 |
| 13 | 10 | 0.04 | 0.02 | 1.7 | 198 |
| 15 | 10 | 0.03 | 0.01 | 1.2 | 244 |
| 17 | 12 | 0.02 | 0.01 | 22 | 236 |
| 18 | 12 | 0.03 | 0.01 | 4.2 | 304 |
| 19 | 14 | 0.03 | 0.02 | 36 | 348 |
| 20 | 14 | 0.03 | 0.02 | 0.5 | 292 |
| 21 | 11 | 0.03 | 0.03 | 13 | 348 |
| 22 | 11 | 0.05 | 0.02 | 0.7 | 376 |
| 23 | 12 | 0.04 | 0.018 | 0.45 | 295 |
| Mean | | 0.03 | 0.02 | 6.2 | 317 |
| SEM | | 0.002 | 0.002 | 2.9 | 18 |

**= p < 00.1
*= p < 0.05 PRL or bGH transgenic compared to control animals
§§= p < 0.01 PRL transgenic compared to bGH transgenic

TABLE 2
Expression of the rat prolactin transgene, the mouse
prolactin gene and the prolactin receptor in the prostate
measured by RT-PCR as described in materials and methods.
Different lobes of the prostate gland were collected at
14 to 15 months of age

| | | Transgenic | Prolactin | Mouse |
|---|---|---|---|---|
| Animal | Prostate lobe | rat prolactin | receptor | prolactin |
| Transgenic animals | | | | |
| L1 | Dorso-lateral | ++ | ++ | na |
| | Ventral | ++ | ++ | na |
| L2 | Dorso-lateral | ++ | ++ | na |
| | Ventral | ++ | ++ | na |
| L3 | Dorso-lateral | + | ++ | na |
| | Ventral | + | ++ | na |
| Normal animals | | | | |
| Normal | Dorso-lateral | na | ++ | ++ |
| | Ventral | na | ++ | ++ |

na = not analyzed

### Example 2

Cultivation of cells from transgenic animals

An expression vector consisting of the rat Pr1 gene driven by a metallothionens promoter was used in transgenic animals. One of the phenotypic alterations was the occurrence of breast cancer in transgenic animals. Below the applicants describe that mammary glands from rPr1 transgenics can be cultivated in vitro either as explants or as established cell lines. It is to be noted that cells modified by transgenic expression behaved differently compared to other mammary gland cells; the cells continued to express Pr1 and attempts to differentiate breast cancer cells, a process that normally require the action of Pr1, did not require Pr1 because the transgene had taken over this function.

Material and methods

2A—Organ culture

Abdominal mammary glands from 9 months old rPRL transgenic mice and control mice at gestation day 14 were dissected out and placed in ice-cold Hanks' balanced salt solution. After gently cutting the glands into small pieces, the explants were transferred into tissue culture dishes. As shown in Table 2A, a subset of cells were exposed to hormonal treatment; dexametasone 1 mM, insulin 5 mg/ml and PRL 5 mg/ml.

After 7 days in culture the explants were processed for histological examination.

TABLE 2A

| Group | Mammary tissue | Hormonal treatment |
|---|---|---|
| 1 | rPRL-trans | no treatment |
| 2 | rPRL-trans | insulin, dexametasone, PRL |
| 3 | rPRL-trans | insulin, dexametasone |
| 4 | control | insulin, dexametasone |
| 5 | rPRL-trans and control | insulin, dexametasone |

Results

After culture in presence of insulin and dexametasone, mammary gland explants from rPRL transgenic mice started to differentiate and to produce milk in contrast to normal mammary gland explants which do not differentiate in absence of PRL.

During co-culture between normal and rPRL transgenic mammary explants in the presence of insulin and dexametasone the normal explant starts to differentiate as well as the transgenic explants.

Conclusion

The expression of rPRL in the mammary gland from rPRL transgenic animals can substitute for PRL in the culture medium indicating the biological significance of the locally PRL expressed in the mammary gland. Such cells could be used in a procedure to screen for compounds that interfere with the actions of Pr1.

2B—Establishment of cellines from mammary tumors in rPRL transgenic mice

A mammary tumor from a rPRL transgenic female mice was dissected out and gently cut into small pieces. The pieces were either treated in Collagenase 8 mg/10 ml for 15 min in 37 C or stayed untreated before cultured.

Dulbecco's MEM nut mix F-12 medium was used with 15% FCS, fungizone 500 mg/l, gentamycin sulfate 50 mg/l, L-glutamine 2 mmol/l and L-ascorbic acid 100 mg/l in a humidified 5% CO2 atmosphere at 37° C.

Results

Both cellines established produced rat-PRL detectable by RIA in the cell culture media and such cells are useful to discover new compounds e.g. by screening compound libraries, that inhibit or facilitate the action of the transgene.

### Example 3

Generation of transgenic animals that are “humanized” in terms of GH receptor expression

A cDNA construction was made that contained the extracellular part of the human GH receptor and the intracellular part of the rat GH receptor. A transgene was subsequently made that consisted of a metallothionein promoter, a signal sequence from human GH the extracellular part of the hGH receptor fused to the intracellular rat GH receptor at a common Nco 1 site. The exchange of signal sequence is of significance for its function in transgenic animals.

BRL (buffalo rat liver) cells were transfected with an expression vector containing the GH receptor cDNA as described above together with a plasmid for neomycin selection. A single cell clone was selected and used for the reporter gene assay. A reporter gene consisting of STAT5 DNA response elements, a TK promoter and a luciferase gene was transfected into the cells (using DOTAB, Boeringer Mannheim) after a 12 h incubation, the media was changed and a subset of cells were exposed to GH during an over-night incubation. Luciferase activity was subsequently measured in cell extracts..

The techniques for cell transfection and the design of the GH reporter gene has been described by Wood et al (Wood et al. J. Biol Chem. 129: 9448-9453 (1995). The transgene construct was also used to generate transgenic mica. A 4.2 kbp fragment was cleaved, purified and injected into fertilized CBA/B6 oocytes. Transgenic offspring were identified by polymerase chain reaction (PCR) using primers complementary to the 5′ part of the metallothionein promoter and the 3′ part of the intracellular domain of the rat GH receptor. 6 mice were found to contain the full length construct and 5 of these transmitted the transgene to offspring.

Table 3 shows that this DNA construction activates a GH dependent reporter gene when stably transfected into BRL cells and similar results were obtained in COS cells (not shown).

TABLE 3
Function of a GH receptor cDNA consisting of the extra-
cellular part of the human GH receptor and the intra-
cellular part of the rGH receptor

| Treatment | Reporter gene activity (light units) |
|---|---|
| Vehicle | 10 195 +/− 1080 |
| GH (10 nM) | 40.645 +/− 6025 |

Conclusion

These results show that non-human cells can be made to respond to GH through the expression of a human GH receptor cDNA variant and that such a gene can be incorporated and inherited in transgenic animals. Alternative ways to achieve expression of human GH receptors is e.g. to use larger receptor constructs that contain the entire hGH receptor gene locus, e.g. on a P1 or on a YAC clone (not shown). The existence of these animals allow the testing of compounds that activate or inhibit the expressed receptor.

1. A method for the screening and identification of low molecular weight compounds which interact with lactogenic/somatogenic receptors, characterized by the use of isolated tissues for in vitro cultivation as organ culture, primary cells, immortalized or transformed cells from transgenic non-human animal that over-express hormones/receptors belonging to the somatogenic or lactogenic family.

2. A method according to claim 1 in which the transgenic non-human animal is used for screening and identifying of substances that influence the phenotypes induced by transgenic over-expression of somatogenic/lactogenic hormones or corresponding receptors

3. A method according to any of claims 1 or 2 in which the utilized tissues derived from transgenic non-human animals have been modified to; (1) withstand in vitro culture conditions and optionally (2) contain a new gene element, a reporter gene, that is characterized by an ability to react to signals activated by the transgene.

4. A method according to claim 3 in which the introduction of the new gene element could be achieved by cellular transfection or by cross-breeding of mice; one that express the reporter gene and the other that over-express the lactogenic hormone.

5. A method according to any of claims 1 to 4 in which cells are used to screen for substances that interfere with the expressed transgene or pathways activated by the same.

6. A method according to claim 5 in which end point measurement parameters are changes in cell proliferation, cell morphology, gene expression, cell signaling molecules, receptor internalization, effector molecules and/or reporter gene activity.

7. A transgenic non-human animal that over-express prolactin which cause phenotypic alterations notably in the prostate and/or mammary gland.

8. A transgenic non-human animal according to claim 7 model which over-expresses human GH receptors.

9. Use of a transgenic non-human animal according to any of claims 7 and 8 in a method according to any of claims 1-6.

10. Use of a transgenic non-human animal according to any of claims 7 and 8 for testing of inhibitory substances that reverse the phenotype induced by transgenic over-expression of prolactin.

11. Use of a transgenic non-human animal according to claim 8 in a method according to claims 1-6 for testing of substances that interfere specifically with the human receptor.
